(19) United States
(12) Patent Application Publication
(10) Pub. No.: US 2021/0330218 A1
(43) Pub. Date: Oct. 28, 2021

(54) MULTI-FACTOR ACTIVITY MONITORING

(71) Applicant: GLYMPSE BIO, INC., Cambridge, MA (US)

(72) Inventors: James Bowen, Cambridge, MA (US); Faycal Touti, Cambridge, MA (US)

(21) Appl. No.: 17/238,593

(22) Filed: Apr. 23, 2021

Related U.S. Application Data
(60) Provisional application No. 63/015,342, filed on Apr. 24, 2020.

(51) Int. Cl. A61B 5/1473; A61B 5/00; G16H 40/67; G16H 10/60; G16H 20/10; G06N 20/00

(52) U.S. Cl. CPC A61B 5/1473; A61B 5/7264; A61B 5/4842; G06N 20/00; G16H 40/67; G16H 10/60; G16H 20/10; A61B 5/4848

(57) ABSTRACT

Data from activity sensors sensitive to enzymes indicative of various disease states is combined with data from other sources including electronic medical records and clinical data including molecular diagnostic testing. The pooled data can be analyzed to identify patterns indicative of certain outcomes including development of a disease, progression of a disease, or likely therapeutic efficacy of a given treatment for a patient.

### TECHNICAL FIELD

The invention relates to multi-factor personalized medicine including non-invasive activity sensors.

### BACKGROUND

Current approaches to detecting or diagnosing diseases such as cancer involve techniques such as obtaining a tissue biopsy and examining cells under a microscope or sequencing DNA to detect genetic markers of the disease. Early detection is advantageous because some treatments will have a greater chance of success with early intervention. For example, with cancer, a tumor may be surgically removed and a patient may go into full remission if the cancer is detected before it metastasizes.

Unfortunately, existing approaches to disease detection do not always detect a disease at its incipiency. For example, while x-ray mammogram represents an advance over manual examination in that an x-ray may detect a tumor that cannot be detected by physical examination. Such tests nevertheless require a tumor to have progressed to some degree for detection to occur. Liquid biopsy represents one potential method for disease detection. In a liquid biopsy, a blood sample is taken and screened for small fragments of tumor DNA. Unfortunately, x-ray mammogram, microscopic examination of tissue samples, and liquid biopsy only detect disease that has advance to some degree and do not always detect disease as early as would be most medically beneficial.

### SUMMARY

The invention provides non-invasive detection of enzyme activity to serve as synthetic biomarkers indicative of various health risks, diagnoses, prognoses, and therapeutic susceptibility and response. Differential expression of various enzymes as reported by engineered sensors can be combined with additional sources of information including other clinical assay data (e.g., genomic, proteomic, and epigenetic information) and data from electronic medical records (EMR) to non-invasively provide a variety of diagnostic and prognostic data points. Additional data points including comorbidities, DNA methylation, and telomeric information can be analyzed as well as associated known outcome information in order to identify data patterns that correlate with various outcomes. Data patterns may be identified that are indicative of the presence of or an increased risk of developing cancer or other diseases. Patterns may be tied to other outcomes such as disease progression and therapeutic susceptibility and response including localized immune system activity, and immuno-therapeutic response.

Systems and methods of the invention are particularly suited to identifying new diagnostic and prognostic links between patient data and outcomes. Accordingly engineered sensors sensitive to all serum proteases can be used to comb for new links between differential expression and disease. In addition to general enzyme expression information (e.g., serum proteases), targeted activity sensors such as tumor-localized activity sensors can be used with cleavable reporters sensitive to, for example, immunological enzymes to detect immune responses including induced immuno-therapeutic responses. The additional depth of data afforded from general enzyme information, genomic, proteomic, epigenetic, EMR, and other sources provides new opportunities for the identification of patterns indicative of disease risk, disease progression, and predicted or actual therapeutic response.

Additional data sources contemplated in multi-factor systems and methods of the invention include molecular diagnostic information and EMR data. Relevant molecular diagnostic data can include patient DNA sequences, DNA methylation data, RNA analysis, epigenetics through gene expression profiling, protein analysis. EMR data can include patient medical history, insurance claims patterns, family history, demographic information or any other information obtained from a patient's electronic medical records. Information from any data source can be combined with activity sensor information to determine a disease risk, track disease progression or therapeutic efficacy, or develop a personalized course of treatment based on predicted outcomes in similar patients.

In certain embodiments, multiplexed activity sensor information can be combined with molecular diagnostic information, EMR data, and known outcomes to train machine learning algorithms to identify correlations or patterns between patient characteristics and certain outcomes (e.g.,. development of disease, progression of disease, or response to various treatments). After training such algorithms, patient data can be analyzed for similar patterns in order to diagnose disease or identify a personalized treatment plan most likely to succeed.

Machine learning and artificial intelligence systems provide the benefit of identifying patterns and correlations in data that would generally escape human detection. Accordingly, when more data is provided for analysis, more and tighter correlations can be identified. In the context of medical diagnostics, prognostics, and treatment, new correlations between patient data and disease risk, outcomes, and therapeutic results can reduce treatment times, lead to earlier diagnoses, and save lives. By combining the wealth of information provided via targeted or general activity sensors with existing patient data from molecular diagnostic assays and EMR information, systems and methods of the invention represent an advancement over existing diagnostic techniques.

Activity sensors act as synthetic biomarkers that can be programmed to provide non-invasive reporting of any enzyme level in a specific target tissue through engineering of an enzyme-specific cleavage site in the activity sensor. For example, the activity sensors may be a multi-arm polyethylene glycol (PEG) scaffold linked to four or more polypeptide reporters as the cleavable analytes. The cleavable linkers are specific for different enzymes whose activity is characteristic of a condition to be monitored (e.g., a certain stage or progression in cancerous tissue or an immune response). When administered to a patient, the activity sensors locate to a target tissue, where they are cleaved by the enzymes to release the detectable analytes. The analytes are detected in a patient sample such as a urine sample. The detected analytes serve as a report of which enzymes are active in the tissue and, therefore, the associated condition or activity.

Because enzymes are differentially expressed under the physiological state of interest such as a disease stage or degree of disease progression, analysis of the sample provides a non-invasive test for the physiological state (e.g., disease stage or condition) of the organ, bodily compartment, bodily fluid, or tissue. The carrier structure preferably includes multiple molecular subunits and may be, for example, a multi-arm polyethylene glycol (PEG) polymer, a lipid nanoparticle, or a dendrimer. The detectable analytes may be, for example, polypeptides that are cleaved by proteases that are differentially expressed in tissue or organs under a specified physiological state, e.g., affected by disease. Because the carrier structure and the detectable analytes are biocompatible molecular structures that locate to a target tissue and are cleaved by disease-associated enzymes to release analytes detectable in a sample, compositions of the disclosure provide non-invasive methods for detecting and characterizing a disease state or stage of an organ or tissue. Because the compositions provide substrates that are released as detectable analytes by enzymatic activity, quantitative detection of the analytes in the sample provide a measure of rate of activity of the enzymes in the organ or tissue. Thus methods and compositions of the disclosure provide non-invasive techniques for measuring both stage and rate of progression of a disease or condition in a target organ or tissue.

Additionally, the activity sensors may include molecular structures to influence trafficking of the sensors within the body, or timing of the enzymatic cleavage or other metabolic degradation of the particles. The molecular structures may function as tuning domains, additional molecular subunits or linkers that are acted upon by the body to locate the activity sensor to the target tissue under controlled timing. For example, the tuning domain may modulate the particle's fate by protecting the activity sensor from premature cleavage and indiscriminate hydrolysis, shielding the particle from immune detection and clearance, or by targeting the particle to specific tissue or cell types. Trafficking may be influenced by including additional molecular structures in the core carrier polymer by, for example, increasing a size of a PEG scaffold to slow degradation of the particle in the body.

In certain embodiments, activity sensors and data analyses methods of the invention can be applied to immuno-oncology (I-O) treatments to predict or observe I-O drug responses in patients. By providing more detailed and relevant information regarding individual patients, new patterns may be identified among responders and non-responders in trials and the information obtained via the activity sensors can be combined with EMR data and molecular diagnostic information for better patient stratification during clinical trials and may help identify patient subpopulations that stand to benefit from specific treatments. Accordingly, systems and methods of the invention may support the clearance of helpful therapies that would have previously been discarded based on limited understanding of patient characteristics relevant to responsiveness or adverse effects.

As noted herein, activity sensors may include a variety of different cleavable reporters sensitive to different enzymes. Furthermore many different activity sensors can be administered and analyzed simultaneously. The reporter molecules can be distinguishable from one another such that multiplex analysis of a variety of protease activities can be accomplished, painting a more detailed picture of the target environment than previously possible using natural biomarkers.

In certain embodiments activity sensor data may be collected periodically along with molecular diagnostic information and other data such as EMR information to provide a chronological tracking of changes in various data points. In addition to point-in-time information, the rate of change in those data points can be examined to provide velocity information. Such information is useful for providing an indication of health, which is applicable even to healthy individuals and provides another data point beyond traditional longitudinal monitoring for disease progression and therapeutic response.

Other data sources may include, for example, medical records, claims data, and test results. Medical records and claims data can provide demographic data, geographic data, medical history, genetic data, laboratory and laboratory test results. Molecular diagnostic data sources may include, for example, RNA expression information or genomic analyses/sequencing data.

Machine learning systems used in the invention can be fully autonomous, i.e., not requiring human input in annotating or labelling data features. Instead, only raw data and associated outcomes are provided to the machine learning system. The machine learning system is then free to identify any features or series of features or feature relationships that are common in data obtained from patients with a certain outcome (e.g., a disease diagnosis, responsiveness to a certain treatment, or disease progression) and therefore indicative of that outcome. The identified feature or features can then be used to predict a patient outcome based on activity sensor, EMR, molecular diagnostic, and other data in new patients with unknown outcomes. More accurate diagnoses and prognoses can thereby be provide in patients of unknown disease status.

A benefit of machine learning analysis is the identification of features or patterns of features that may be used to predict outcome without the need to understand any underlying relationship between the disease and the identified feature. Accordingly, identified correlations can be studied to better understand disease mechanisms. Machine learning systems of the invention may use or include, for example, one or more of a neural network, a random forest, regression analysis, a support vector machine (SVM), cluster analysis, decision tree learning, association rule learning, or a Bayesian network.

In various embodiments the activity sensor carrier structure can include multiple molecular subunits and may be, for example, a multi-arm polyethylene glycol (PEG) polymer, a lipid nanoparticle, or a dendrimer. The detectable analytes may be, for example, polypeptides that are cleaved by proteases that are differentially expressed in tissue or organs experiencing an immune response or undergoing a disease progression. Because the carrier structure and the detectable analytes are biocompatible molecular structures that locate to a target tissue and are cleaved by disease or immune-response-associated enzymes to release analytes detectable in a sample, compositions of the disclosure provide non-invasive methods for detecting and characterizing the state of an organ or tissue. Because the compositions provide substrates that are released as detectable analytes by enzymatic activity, quantitative detection of the analytes in the sample provide a measure of rate of activity of the enzymes in the organ or tissue. Thus methods and compositions of the disclosure provide non-invasive techniques for measuring both stage and rate of progression of cancer or response to I-O therapies.

Activity sensors may take the form of cyclic peptides that are naturally resistant to off-target degradation. The target environment may be a tumor microenvironment in which a specific enzyme or set of enzymes are differentially-expressed. A cyclic peptide may be engineered with cleavage sites specific to enzymes in the tumor (e.g., unique enzymes expressed preferentially in the tumor). The engineered peptide, in its cyclic form, can travel through the blood and other potentially harsh environments protected against degradation by common non-specific proteases and without interacting in a meaningful way with off-target tissues. Only upon arrival within the specific target tissue and exposure to the required enzyme or combination of enzymes, the cyclic peptide is cleaved to produce a linear molecule that is capable of clearance and sample observation. For purposes of the application and as will be apparent upon consideration of the detailed description thereof, a linear peptide is any peptide that is not cyclic. Thus, for example, a linearized peptide may have various branch chains.

Cyclic peptides can be engineered with other cleavable linkages, such as ester bonds in the form of cyclic depsipeptides in which the degradation of the ester bond releases a linearized peptide ready to react with its target environment. Thioesters and other tunable bonds can be included in the cyclic peptide to create a timed-release in plasma or other environments. See Lin and Anseth, 2013 Biomaterials Science (Third Edition), pages 716-728, incorporated herein by reference.

Macrocyclic peptides may contain two or more protease-specific cleavage sequences and can require two or more protease-dependent hydrolytic events to release a reporter peptide or a bioactive compound. The protease-specific sequences can be different in various embodiments. In cases where cleavage of multiple sites is required to release the linearized peptide, different protease-specific sequences can increase specificity for the release as the presence of at least two different target-specific enzymes will be required. The specific and non-specific proteolysis susceptibility and rate can be tuned through manipulation of peptide sequence content, length, and cyclization chemistry.

Activity sensors may include additional molecular structures to influence trafficking of the peptides within the body, or timing of the enzymatic cleavage or other metabolic degradation of the particles. The molecular structures may function as tuning domains, additional molecular subunits or linkers that are acted upon by the body to locate the activity sensor to the target tissue under controlled timing. For example, the tuning domain may target the particle to specific tissue or cell types. Trafficking may be influenced by the addition of molecular structures in the carrier polymer by, for example, increasing the size of a PEG scaffold to slow degradation in the body.

In certain embodiments, the invention provides a tunable activity sensor that reveals enzymatic activity associated with a physiological state, such as disease. When the activity reporter is administered to a patient, it is trafficked through the body to specific cells or specific tissues. For example, in a patient with lung cancer, the activity sensor may be tuned to localize in the cancerous tissue through, for example, the use of tuning domains preferentially trafficked to lung tissue or tumor tissue. The activity sensors can include cleavable reporter molecules sensitive to enzymes indicative to an immune response or a stage of tumor progression or regression. Subsequent observation and/or tracking of reporter levels in a patient sample (e.g., urine) will then provide an indication of the progression and/or therapeutic response of the patient's lung cancer.

The sensor may be designed or tuned so that it remains in circulation, e.g., in blood, or lymph, or both. If enzymes that are differentially expressed under conditions of a particular disease are present, those enzymes cleave the reporter and release a detectable analyte. Cyclic peptide activity sensors may be used to resist non-specific degradation of the peptide in circulation while still providing an accessible substrate for cleavage by the target proteases.

Molecular structures can be included in the activity sensor as tuning domains, to tune or modify a distribution or residence time of the activity sensor within the subject. The tuning domains may be linked any portion of the activity sensor and may be modified in numerous ways. Through the use of tuning domains, one may modify the activity sensor's distribution within the body depending on in vivo trafficking pathways to a specific tissue, or its residence time within systemic circulation or within a specific tissue. Additionally, the tuning domains may promote effective cleavage of the reporter by tissue-specific enzymes or prevent premature cleavage or hydrolysis. Because the detectable analytes are the product of enzymatic activity and the activity sensors can be provided in excess, the signal given by the analyte is effectively amplified, and the presence of even very small quantity of active enzyme may be detected.

Aspects of the invention include methods of monitoring cancer progression including administering to a patient suspected of having cancer an activity sensor comprising a carrier linked to a reporter molecule by a cleavable linker containing the cleavage site of an enzyme indicative of a characteristic of a tumor environment. A sample such as a urine sample can be collected from the patient and analyzed to detect the presence or lack of the reporter, where presence of the reporter is indicative of the characteristic.

The characteristic may be an active immune response and the patient is undergoing immuno-oncological treatment, wherein presence of the reporter is indicative of therapeutic effect of the immuno-oncological treatment. The activity sensor may include a tuning domain operable to localize the activity sensor in a target tumor. The characteristic can be a checkpoint inhibited immune response and, wherein presence of the reporter is indicative of a predicted therapeutic response to a checkpoint inhibitor therapy. Methods may include stratifying the patient in a clinical trial based on the detection of the reporter in the sample.

In certain embodiments, the analyzing step may include quantifying a level of the reporter in the sample and the method can include periodically repeating the administering, collecting, and analyzing steps to prepare a chronological series of reporter levels from which a velocity of the characteristic can be determined that is indicative of cancer progression in the patient.

### BRIEF DESCRIPTION OF THE DRAWINGS

FIG. 1 diagrams steps of a method for analyzing patient data.

FIG. 2 shows an activity sensor.

FIG. 3 shows an engineered macrocyclic peptide.

FIG. 4 shows a schematic of the computational analysis platform.

### DETAILED DESCRIPTION

The invention provides activity sensors that non-invasively provide detailed information on the differential expression of enzymes in patient tissues. That information is combined with data from other sources such as clinical data (e.g., molecular diagnostic testing), and information from electronic medical records (EMR) to provide numerous patient-specific data points. The combination of large amounts of data allows for new diagnostic, prognostic, and therapeutic indicators to be identified in order improve patient outcomes. In certain embodiments, data analysis is conducted by machine learning systems to identify correlations between various data points and patient outcomes (e.g., treatment responsiveness and development or progression of disease). Activity sensors can include a variety of reporter molecules that are detectable in a body fluid sample such as urine but are only released from the body upon cleavage by a specific enzyme or group of enzymes. Accordingly, detection of the reporters in the sample is indicative of the differential expression of the enzymes in the target tissue. In certain embodiments, a wide-ranging cocktail of activity sensors can be administered to report on expression data of all serum proteases. In addition to general serum protease expression, by targeting the activity sensors to specific tissues (e.g., tumors) and engineering their cleavage sites to be specific to enzymes differentially expressed under various conditions, activity sensors of the invention can provide insight into disease progression and predicted or actual therapeutic response.

Activity sensors and data analysis methods of the invention can be applied to treatments to predict or observe drug responses in patients. The depth of information provided from the combination of activity sensors, clinical data, and EMR information can offer new factors for use in patient stratification for clinical trials, for example. Stratification is the partitioning of subjects and results by a factor other than the treatment given. Stratification is traditionally done by factors such as gender, age, or other demographic details but the addition of detailed patient information obtained via activity sensors and other clinical testing can provide more practical and meaningful groups for stratification. Examining patient responses in view of such groupings can be used to eliminate variables to better interpret results and map adverse events or therapeutic efficacy to causative patient characteristics.

Activity sensors act as synthetic biomarkers that can be programmed to provide non-invasive reporting of any enzyme level in a specific target tissue through engineering of an enzyme-specific cleavage site in the activity sensor. When administered to a patient, the activity sensors locate to a target tissue using, for example, target-specific tuning domains. Once localized, they are cleaved by the enzymes to release the detectable analytes. The analytes are detected in a patient sample such as a urine sample. The detected analytes serve as a report of which enzymes are active in the tissue and, therefore, the associated condition or activity. Localization allows activity sensors to report on the conditions of a target tissue without contamination of off-target information. That ability is useful in differentiating anti-tumor immune responses indicative of successful I-O treatment from an off-target immune response that may, for example, be occurring in response to a viral infection.

Additionally, because activity sensor monitoring, many genomic and RNA expression studies, and EMR data analysis does not require invasive operations, frequent monitoring is more feasible and up-to-date information on disease progression and therapeutic response allows for quicker decisions for assessing safety and efficacy. For example, frequent monitoring can be used to quickly identify resistances to treatment as they develop. For example, as cancers progress, they continue to mutate and neo-antigens used to target immunotherapies may no longer be expressed, causing therapeutic effectiveness to diminish. The ability to quickly identify such changes through monitoring with activity sensors and other EMR and clinical data can lead to faster therapy changes, perhaps before significant cancer progression or recurrence.

Enzyme-specific reporters can be multiplexed on single activity sensors or in many different activity sensors that are administered and analyzed simultaneously. The reporter molecules can be specific for each enzyme such that they can be distinguished in multiplex analysis. In certain embodiments, activity sensors, acting as synthetic biomarkers, may be administered and measured periodically. The changes in enzyme levels over time can be examined along with changes in other clinical or EMR data to provide a chronological mapping of data points. Studies have found that biomarker velocity (the rate of change in biomarker levels over time) may be a better indicator of disease progression (or regression) than any single threshold. The same principle can be applied to the activity sensors of the invention acting as synthetic biomarkers.

Activity sensors can include a carrier, at least one reporter linked to the carrier and at least one tuning domain that modifies a distribution or residence time of the activity sensor within a subject when administered to the subject. The activity sensor may be designed to detect and report enzymatic activity in the body, for example, enzymes that are differentially expressed during immune responses or during tumor progression or regression. Dysregulated proteases have important consequences in the progression of diseases such as cancer in that they may alter cell signaling, help drive cancer cell proliferation, invasion, angiogenesis, avoidance of apoptosis, and metastasis.

The activity sensor may be tuned via the tuning domains in numerous ways to facilitate detecting enzymatic activity within the body in specific cells or in a specific tissue. For example, the activity sensor may be tuned to promote distribution of the activity sensor to the specific tissue or to improve a residence time of the activity sensor in the subject or in the specific tissue. Tuning domains may include, for example, molecules localized in rapidly replicating cells to better target tumor tissue.

When administered to a subject, the activity sensor is trafficked through the body and may diffuse from the systemic circulation to a specific tissue, where the reporter may be cleaved via enzymes indicative of disease presence or progression. The detectable analyte may then diffuse back into circulation where it may pass renal filtration and be excreted into urine, whereby detection of the detectable analyte in the urine sample indicates enzymatic activity in the target tissue.

The carrier may be any suitable platform for trafficking the reporters through the body of a subject, when administered to the subject. The carrier may be any material or size suitable to serve as a carrier or platform. Preferably the carrier is biocompatible, non-toxic, and non-immunogenic and does not provoke an immune response in the body of the subject to which it is administered. The carrier may also function as a targeting means to target the activity sensor to a tissue, cell or molecule. In some embodiments the carrier domain is a particle such as a polymer scaffold. The carrier may, for example, result in passive targeting to tumors or other specific tissues by circulation. Other types of carriers include, for example, compounds that facilitate active targeting to tissue, cells or molecules. Examples of carriers include, but are not limited to, nanoparticles such as iron oxide or gold nanoparticles, aptamers, peptides, proteins, nucleic acids, polysaccharides, polymers, antibodies or antibody fragments and small molecules.

The carrier may include a variety of materials such as iron, ceramic, metallic, natural polymer materials such as hyaluronic acid, synthetic polymer materials such as poly-glycerol sebacate, and non-polymer materials, or combinations thereof. The carrier may be composed in whole or in part of polymers or non-polymer materials, such as alumina, calcium carbonate, calcium sulfate, calcium phosphosilicate, sodium phosphate, calcium aluminate, and silicates. Polymers include, but are not limited to: polyamides, polycarbonates, polyalkylenes, polyalkylene glycols, polyalkylene oxides, cellulose ethers, cellulose esters, nitro celluloses, polymers of acrylic and methacrylic esters, methyl cellulose, ethyl cellulose, and hydroxypropyl cellulose. Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, poly-anhydrides, polyurethanes, and natural polymers such as alginate and other polysaccharides including dextran and cellulose, collagen, albumin and other proteins, copolymers and mixtures thereof. In general, these biodegradable polymers degrade either by enzymatic hydrolysis or exposure to water in vivo, by surface or bulk erosion. These biodegradable polymers may be used alone, as physical mixtures (blends), or as co-polymers.

In preferred embodiments, the carrier includes biodegradable polymers so that whether the reporter is cleaved from the carrier, the carrier will be degraded in the body. By providing a biodegradable carrier, accumulation and any associated immune response or unintended effects of intact activity sensors remaining in the body may be minimized.

Other biocompatible polymers include PEG, PVA and PVP, which are all commercially available. PVP is a non ionogenic, hydrophilic polymer having a mean molecular weight ranging from approximately 10,000 to 700,000 and has the chemical formula (C6H9NO)[n]. PVP is also known as poly[1 (2 oxo 1 pyrrolidinyl)ethylene]. PVP is nontoxic, highly hygroscopic and readily dissolves in water or organic solvents.

Polyvinyl alcohol (PVA) is a polymer prepared from polyvinyl acetates by replacement of the acetate groups with hydroxyl groups and has the chemical formula (CH2CHOH)[n]. Most polyvinyl alcohols are soluble in water.

Polyethylene glycol (PEG), also known as poly(oxyethylene) glycol, is a condensation polymer of ethylene oxide and water. PEG refers to a compound that includes repeating ethylene glycol units. The structure of PEG may be expressed as H—(O—CH2-CH2)n-OH. PEG is a hydrophilic compound that is biologically inert (i.e., non-immunogenic) and generally considered safe for administration to humans.

When PEG is linked to a particle, it provides advantageous properties, such as improved solubility, increased circulating life, stability, protection from proteolytic degradation, reduced cellular uptake by macrophages, and a lack of immunogenicity and antigenicity. PEG is also highly flexible and provides bio-conjugation and surface treatment of a particle without steric hindrance. PEG may be used for chemical modification of biologically active compounds, such as peptides, proteins, antibody fragments, aptamers, enzymes, and small molecules to tailor molecular properties of the compounds to particular applications. Moreover, PEG molecules may be functionalized by the chemical addition of various functional groups to the ends of the PEG molecule, for example, amine-reactive PEG (BS (PEG)n) or sulfhydryl-reactive PEG (BM (PEG)n).

In certain embodiments, the carrier is a biocompatible scaffold, such as a scaffold including polyethylene glycol (PEG). In a preferred embodiment, the carrier is a biocompatible scaffold that includes multiple subunits of covalently linked polyethylene glycol maleimide (PEG-MAL), for example, an 8-arm PEG-MAL scaffold. A PEG-containing scaffold may be selected because it is biocompatible, inexpensive, easily obtained commercially, has minimal uptake by the reticuloendothelial system (RES), and exhibits many advantageous behaviors. For example, PEG scaffolds inhibit cellular uptake of particles by numerous cell types, such as macrophages, which facilitates proper distribution to a specific tissues and increases residence time in the tissue.

An 8-arm PEG-MAL is a type of multi-arm PEG derivative that has maleimide groups at each terminal end of its eight arms, which are connected to a hexaglycerol core. The maleimide group selectively reacts with free thiol, SH, sulfhydryl, or mercapto group via Michael addition to form a stable carbon sulfur bond. Each arm of the 8-arm PEG-MAL scaffold may be conjugated to peptides, for example, via maleimide-thiol coupling or amide bonds.

The PEG-MAL scaffold may be of various sizes, for example, a 10 kDa scaffold, a 20 kDa scaffold, a 40 kDa scaffold, or a greater than 40 kDa scaffold. The hydrodynamic diameter of the PEG scaffold in phosphate buffered saline (PBS) may be determined by various methods known in the art, for example, by dynamic light scattering. Using such techniques, the hydrodynamic diameter of a 40 kDa PEG-MAL scaffold was measured to be approximately 8 nm. In preferred embodiments, a 40 kDa PEG-MAL scaffold is provided as the carrier when the activity sensor is administered subcutaneously because the activity sensor readily diffuses into systemic circulation but is not readily cleared by the reticuloendothelial system.

The size of the PEG-MAL scaffold affects the distribution and residence time of the activity sensor in the body because particles smaller than about 5 nm in diameter are efficiently cleared through renal filtration of the body, even without proteolytic cleavage. Further, particles larger than about 10 nm in diameter often drain into lymphatic vessels. In one example, where a 40 kDa 8-arm PEG-MAL scaffold was administered intravenously, the scaffold was not renally cleared into urine.

The reporter may be any reporter susceptible to an enzymatic activity, such that cleavage of the reporter indicates that enzymatic activity. The reporter is dependent on enzymes that are active in a specific disease state. For example, tumors are associated with a specific set of enzymes. For a tumor, the activity sensor may be designed with an enzyme susceptible site that matches that of the enzymes expressed by the tumor or other diseased tissue. Alternatively, the enzyme-specific site may be associated with enzymes that are ordinarily present but are absent in a particular disease state. In this example, a disease state would be associated with a lack of signal associated with the enzyme, or reduced levels of signal compared to a normal reference or prior measurement in a healthy subject.

In various embodiments, the reporter includes a naturally occurring molecule such as a peptide, nucleic acid, a small molecule, a volatile organic compound, an elemental mass tag, or a neoantigen. In other embodiments, the reporter includes a non-naturally occurring molecule such as D-amino acids, synthetic elements, or synthetic compounds. The reporter may be a mass-encoded reporter, for example, a reporter with a known and individually-identifiable mass, such as a polypeptide with a known mass or an isotope.

An enzyme may be any of the various proteins produced in living cells that accelerate or catalyze the metabolic processes of an organism. Enzymes act on substrates. The substrate binds to the enzyme at a location called the active site before the reaction catalyzed by the enzyme takes place. Generally, enzymes include but are not limited to proteases, glycosidases, lipases, heparinases, and phosphatases. Examples of enzymes that are associated with disease in a subject include but are not limited to MMP, MMP-2, MMP-7, MMP-9, kallikreins, cathepsins, seprase, glucose-6-phosphate dehydrogenase (G6PD), glucocerebrosidase, pyruvate kinase, tissue plasminogen activator (tPA), a disintegrin and metalloproteinase (ADAM), ADAMS, ADAM15, and matriptase. The detected enzymatic activity may be activity of any type of enzyme, for example, proteases, kinases, esterases, peptidases, amidases, oxidoreductases, transferases, hydrolases, lysases, isomerases, or ligases.

Examples of substrates for disease-associated enzymes include but are not limited to Interleukin 1 beta, IGFBP-3, TGF-beta, TNF, FASL, HB-EGF, FGFR1, Decorin, VEGF, EGF, IL2, IL6, PDGF, fibroblast growth factor (FGF), and tissue inhibitors of MMPs (TIMPs).

Systems and methods of the invention may be used to monitor cancer progression or predict or monitor treatment response to an immuno-oncological therapy through the measurement of immunological enzyme levels combined with other data. Enzymes indicative of immune response can include, for example, tissue remodeling enzymes. Several proteases are known to be associated with inflammation and programmed cell death (e.g., including apoptosis, pyroptosis and necroptosis). The localized levels of those proteases is accordingly indicative of immune system activity. Caspases (cysteine-aspartic proteases, cysteine aspartases or cysteine-dependent aspartate-directed proteases) are a family of protease enzymes including a cysteine in their active site that nucleophilically cleaves a target protein only after an aspartic acid residue. Caspase-1, Caspase-4, Caspase-5 and Caspase-11 are associated with inflammation. Serine proteases also function in apoptosis and inflammation and their differential expression is therefore also indicative of an immune response. Immune cells express serine proteases such as granzymes, neutrophil elastase, cathepsin G, proteinase 3, chymase, and tryptase.

In various embodiments, it may be useful to differentiate between programmed cell death indicative of an immune response and necrosis naturally found during tumor progression. In contrast to programmed cell death, where caspases and serine proteases are the primary proteases, calpains and lysosomal proteases (e.g., cathepsins B and D) are the key proteases in necrosis. Accordingly, calpain and cathepsin levels indicated by activity sensor reporter measurements can provide information regarding necrotic cell death to supplement the immuno-oncological information.

Activity sensors and methods of the invention can be applied to I-O treatments to observe I-O drug responses in patients. For example, activity sensors with cleavage sites sensitive to caspases, serine proteases, calpains, and cathepsins can be administered during or after I-O treatment and reporter levels in patient samples can be used to monitor therapeutic response. A baseline signal of caspases or serine proteases in patient samples is indicative of a non-responsive tumor. The baseline level can be determined experimentally through data collected from patient populations or pre-treatment data from the patient undergoing treatment. Increased signals of caspases and serine proteases during or after treatment relative to a baseline level can be indicative of a desired immuno-oncological response. Tracking the levels of calpain or cathepsin signals can provide additional information on non-immunological cell death that may be associated with tumor progression.

The tuning domains may include any suitable material that modifies a distribution or residence time of the activity sensor within a subject when the activity sensor is administered to the subject. For example, the tuning domains may include PEG, PVA, or PVP. In another example, the tuning domains may include a polypeptide, a peptide, a nucleic acid, a polysaccharide, volatile organic compound, hydrophobic chains, or a small molecule.

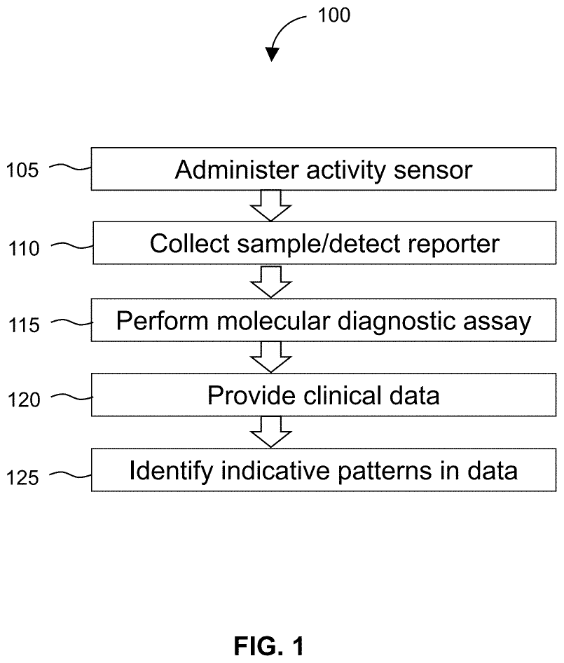

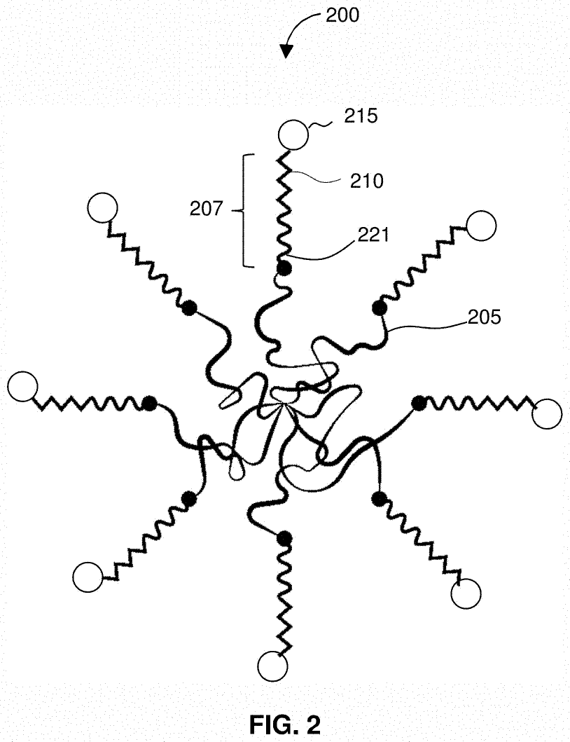

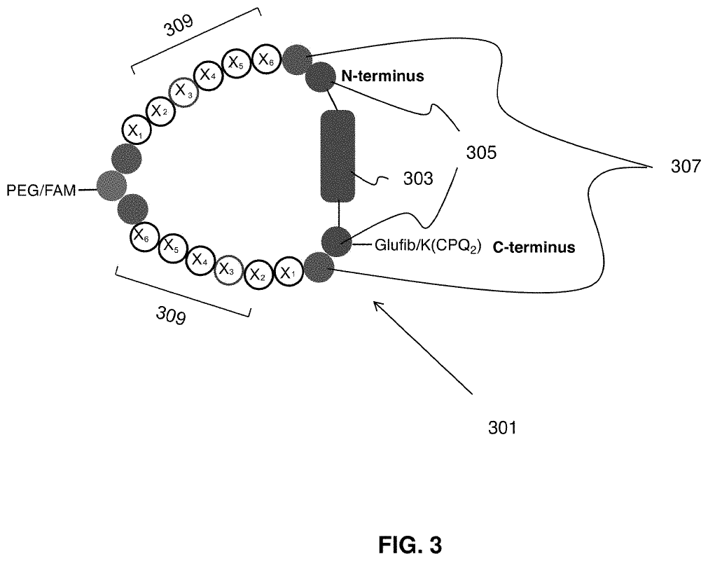

FIG. 1 diagrams steps of a method 100 for analyzing patient data. At step 105, an activity sensor is administered to a patient. The patient may be healthy, suspected of having a disease, known to have a disease, at risk of developing a disease, and/or undergoing treatment. The activity sensor includes a reporter linked by a cleavable linker to a carrier (e.g., as shown in FIGS. 2 and 3). The cleavable linker is sensitive to an enzyme for which the level is indicative of a disease state (e.g., enzymes upregulated in expanding tumors or tumors in regression, or enzymes indicative of active or inhibited immune responses). As discussed herein, depending on the enzyme activity the activity sensors are engineered to report on and the patient's disease and treatment status, information garnered from reporter levels in patient samples can be used to diagnose and/or stage the disease, monitor progression, predict responsiveness to a given therapy, and monitor therapeutic effectiveness. Activity sensors can be administered by any suitable method. In preferred embodiments, the activity sensor is delivered intravenously or aerosolized and delivered to the lungs, for example, via a nebulizer. In other examples, the activity sensor may be administered to a subject transdermally, intradermally, intraarterially, intralesionally, intratumorally, intracranially, intraarticularly, intratumorally, intramuscularly, subcutaneously, orally, topically, locally, inhalation, injection, infusion, or by other method or any combination known in the art (see, for example, Remington's Pharmaceutical Sciences (1990), incorporated by reference).

At step 110, after administration of the activity sensor and localization of the activity sensor in the target tissue, the reporter is selectively released upon cleavage of the linker in the presence of the target enzyme. Localization can be accomplished through the use of tuning domains including moieties preferentially concentrated in the target tissue. Upon release of the reporter, it can be cleared by the body into a fluid capable of non-invasive collection such as urine after transport to the blood stream and renal clearance. The sample, such as a urine sample, can be collected for analysis and the presence and/or levels of the reporter in the sample can be detected.

In various embodiments, a cocktail of activity sensors sensitive to different serum proteases may be administered in order to analyze all differential expression data for outcome-associated patterns. Examples of serum proteases include thrombin, plasmin, and Hageman factor.

At step 115 molecular diagnostic assays can be performed or other clinical data can be gathered. Such data can include blood assays, urinalysis, lipid panels, DNA sequencing, immunoassays, RNA expression analysis, and any other test known to those of ordinary skill in the art.

Of particular interest is genomic data which can be obtained, for example, by conducting an assay on a sample to identify variants present within DNA. The presence of certain single nucleotide polymorphisms (SNPs) or other mutations in various genetic regions or abnormal expression levels of those genetic regions may be indicative of a disease risk, stage, progression, or likelihood of responding to various therapies. Variations that can affect disease can include, for example, SNPs, deletions, insertions, inversions, rearrangements, copy number variations (CNVs), chromosomal microdeletion, genetic mosaicism, karyotype abnormalities, and combinations thereof. Methods of detecting such variations and obtaining genomic data are well known in the art.

In certain embodiments, whole genome sequencing may be performed and the genomic data used in methods of the invention may include a patient's genomic sequence. Methods of performing whole genome sequencing are known in the art.

Epigenetic information can also be obtained or provided for analysis including gene expression levels and DNA methylation information. DNA methylation may be determined through any method known in the art including mass spectrometry, methylation-specific PCR, bisulfate sequencing, methylated DNA immunoprecipitation, and ChIP-on-ChIP.

At step 120, clinical data is provided or obtained. Clinical data contemplated for use in methods of the invention can include medical records, clinical trial data, patient and disease registries, administrative data, insurance claims data, health surveys, and archived laboratory results. Medical records can include electronic clinical data which is created and/or stored at the point of care at a medical facility. That material is sometimes known as an electronic medical record (EMR), as used herein EMR includes administrative and demographic information, diagnosis, treatment, prescription drugs, laboratory tests, physiologic monitoring data, hospitalization, patient insurance, etc. Sources of EMR include individual organizations such as hospitals or health systems. EMR may be accessed through larger collaborations, such as the NIH Collaborator Distributed Research Network, which provides mediated or collaborative access to clinical data repositories by eligible researchers. Additionally, the UW De-identified Clinical Data Repository (DCDR) and the Stanford Center for Clinical Informatics allow for initial cohort identification.

Disease registries exist that provide data for certain chronic conditions such as Alzheimer's Disease, cancer, diabetes, heart disease, and asthma. Such registries can be used to provide information useful in methods of the invention.

Administrative data including hospital discharge data reported to a government agency like AHRQ, or data from the Healthcare Cost & Utilization Project (H-CUP) can be used. In various embodiments, insurance claims data including inpatient, outpatient, pharmacy, and enrollment data can be used for analysis with activity sensor information. Government (e.g., Medicare) and/or commercial health firms can be sources for obtaining insurance claims data.

Another source of information can be health surveys such as the National Center for Health Statistics, Center for Medicare & Medicaid Services Data Navigator, the Medicare Current Beneficiary Survey, National Health & Nutrition Examination Survey (NHANES), The Medical Expenditure Panel Survey (MEPS), or the National Health and Aging Trends Study (NHATS). Clinical data may also be obtained from clinical trials registries and databases such as ClinicalTrials.gov, WHO International Clinical Trials Registry Platform (ICTRP), the European Union Clinical Trials Database, the ISRCTN Registry (BioMed Central), or CenterWatch.

Step 125 includes identifying indicative patterns in the data (including activity sensor data, molecular diagnostic data, and clinical data) to diagnose, stage, evaluate risk, or determine a treatment recommendation for a patient. Identifying indicative patterns can be done in an initial training stage which may use known outcomes and a machine learning system or neural network on a computing device to identify links between data patterns and disease. In certain embodiments, identifying indicative patterns can include the application of identified correlations to test data with unknown outcomes where previously identified patterns indicative of a certain outcome are identified in order to predict that outcome for a test patient.

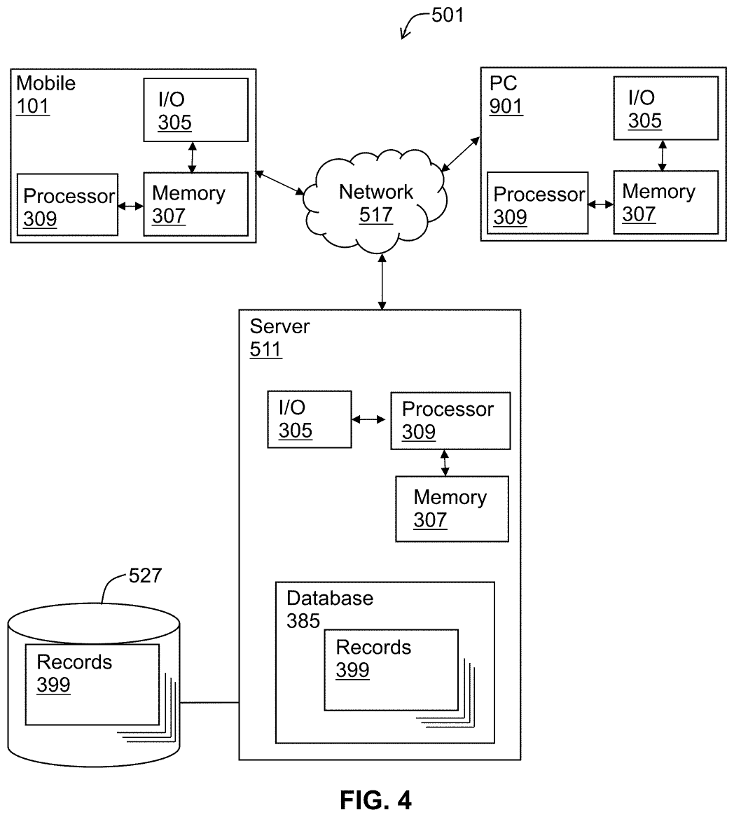

FIG. 4 provides a schematic of computer components that may appear within a computer system 501. System 501 preferably includes at least one server computer system 511 operable to communicate with at least one computing device 101a, 101b via a communication network 517. Sever 511 may be provided with a database 385 (e.g., partially or wholly within memory 307, storage 527, both, or other) for storing records 399 including, for example, patient data, outcomes, or assay results for performing the methodologies described herein. Optionally, storage 527 may be associated with system 501. A server 511 or computing device 101 according to systems and methods of the invention generally includes at least one processor 309 coupled to a memory 307 via a bus and input or output devices 305.

As one skilled in the art would recognize as necessary or best-suited for the systems and methods of the invention, systems and methods of the invention include one or more servers 511 and/or computing devices 101 that may include one or more of processor 309 (e.g., a central processing unit (CPU), a graphics processing unit (GPU), etc.), computer-readable storage device 307 (e.g., main memory, static memory, etc.), or combinations thereof which communicate with each other via a bus.

A processor 309 may include any suitable processor known in the art, such as the processor sold under the trademark XEON E7 by Intel (Santa Clara, Calif.) or the processor sold under the trademark OPTERON 6200 by AMD (Sunnyvale, Calif.).

Memory 307 preferably includes at least one tangible, non-transitory medium capable of storing: one or more sets of instructions executable to cause the system to perform functions described herein (e.g., software embodying any methodology or function found herein); data; or both. While the computer-readable storage device can in an exemplary embodiment be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, hard drives, disk drives, and any other tangible storage media.

Any suitable services can be used for storage 527 such as, for example, Amazon Web Services, memory 307 of server 511, cloud storage, another server, or other computer-readable storage. Cloud storage may refer to a data storage scheme wherein data is stored in logical pools and the physical storage may span across multiple servers and multiple locations. Storage 527 may be owned and managed by a hosting company. Preferably, storage 527 is used to store records 399 as needed to perform and support operations described herein.

Input/output devices 305 according to the invention may include one or more of a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, a button, an accelerometer, a microphone, a cellular radio frequency antenna, a network interface device, which can be, for example, a network interface card (NIC), Wi-Fi card, or cellular modem, or any combination thereof.

One of skill in the art will recognize that any suitable development environment or programming language may be employed to allow the operability described herein for various systems and methods of the invention. For example, systems and methods herein can be implemented using Objective-C, Swift, C, Perl, Python, C++, C#, Java, JavaScript, Visual Basic, Ruby on Rails, Groovy and Grails, or any other suitable tool. For a computing device 101, it may be preferred to use native xCode or Android Java.

Machine learning systems of the invention may be configured to receive activity sensor, molecular diagnostic assay, or clinical data, and known outcomes, to identify features within the data in an unsupervised manner and to create a map of outcome probabilities over the features. The machine learning system can further receive any of the above data from a test subject, identify within the data predictive features learned from the training steps and locate the predictive features on the map of outcome probabilities to provide a prognosis or diagnosis including likely responsiveness to various treatments.

Any of several suitable types of machine learning may be used for one or more steps of the disclosed methods. Suitable machine learning types may include decision tree learning, association rule learning, inductive logic programming, support vector machines (SVMs), and Bayesian networks. Examples of decision tree learning include classification trees, regression trees, boosted trees, bootstrap aggregated trees, random forests, and rotation forests. One or more of the above machine learning systems may be used to complete any or all of the method steps described herein. For example, one model, such as a neural network, may be used to complete the training steps of autonomously identifying features and associating those features with certain outcomes. Once those features are learned, they may be applied to test samples by the same or different models or classifiers (e.g., a random forest, SVM, regression) for the correlating steps. In certain embodiments, features may be identified and associated with outcomes using one or more machine learning systems and the associations may then be refined using a different machine learning system. Accordingly some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. In decision trees, decisions are made sequentially at a series of nodes, which correspond to input variables. Random forests include multiple decision trees to improve the accuracy of predictions. See Breiman, L. Random Forests, Machine Learning 45:5-32 (2001), incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

SVMs are useful for both classification and regression. When used for classification of new data into one of two categories, such as having a disease or not having the disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering to perform unsupervised machine learning suitable for some of the methods discussed herein. See Ben-Hur, A., et al., (2001), Support Vector Clustering, Journal of Machine Learning Research, 2:125-137.

Regression analysis is a statistical process for estimating the relationships among variables such as features and outcomes. It includes techniques for modeling and analyzing relationships between a multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Association rule learning is a method for discovering interesting relations between variables in large databases. See Agrawal, R. et al., “Mining association rules between sets of items in large databases”. Proceedings of the 1993 ACM SIGMOD international conference on Management of data—SIGMOD '93. p. 207 (1993) doi:10.1145/170035.170072, ISBN 0897915925, incorporated herein by reference. Algorithms for performing association rule learning include Apriori, Eclat, FP-growth, and AprioriDP. FIN, PrePost, and PPV, which are described in detail in Agrawal, R. et al., Fast algorithms for mining association rules in large databases, in Bocca, Jorge B.; Jarke, Matthias; and Zaniolo, Carlo; editors, Proceedings of the 20th International Conference on Very Large Data Bases (VLDB), Santiago, Chile, September 1994, pages 487-499 (1994); Zaki, M. J. (2000). “Scalable algorithms for association mining”. IEEE Transactions on Knowledge and Data Engineering. 12 (3): 372-390; Han (2000). “Mining Frequent Patterns Without Candidate Generation”. Proceedings of the 2000 ACM SIGMOD International Conference on Management of Data. SIGMOD '00: 1-12. doi:10.1145/342009.335372; D. Bhalodiya, K. M. Patel and C. Patel. An Efficient way to Find Frequent Pattern with Dynamic Programming Approach [1]. NIRMA UNIVERSITY INTERNATIONAL CONFERENCE ON ENGINEERING, NUiCONE-2013, 28-30 Nov., 2013; Z. H. Deng and S. L. Lv. Fast mining frequent itemsets using Nodesets.[2]. Expert Systems with Applications, 41(10): 4505-4512, 2014; Z. H. Deng, Z. Wang and J. Jiang. A New Algorithm for Fast Mining Frequent Itemsets Using N-Lists [3]. SCIENCE CHINA Information Sciences, 55 (9): 2008-2030, 2012; and Z. H. Deng and Z. Wang. A New Fast Vertical Method for Mining Frequent Patterns [4]. International Journal of Computational Intelligence Systems, 3(6): 733-744, 2010; each of which is incorporated herein by reference.

Inductive logic programming relies on logic programming to develop a hypothesis based on positive examples, negative examples, and background knowledge. See Luc De Raedt. A Perspective on Inductive Logic Programming. The Workshop on Current and Future Trends in Logic Programming, Shakertown, to appear in Springer LNCS, 1999. CiteSeerX:10.1.1.56.1790; Muggleton, S.; De Raedt, L. (1994). “Inductive Logic Programming: Theory and methods”. The Journal of Logic Programming. 19-20: 629-679. doi:10.1016/0743-1066(94)90035-3; incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, E. Bayesian Networks without Tears, AI Magazine, p. 50, Winter 1991.

FIG. 2 shows an activity sensor 200 with carrier 205, reporters 207, and tuning domains 215. As illustrated, carrier 205 is a biocompatible scaffold that includes multiple subunits of covalently linked polyethylene glycol maleimide (PEG-MAL). Carrier 205 is an 8-arm PEG-MAL scaffold with a molecular weight between about 20 and 80 kDa. Reporter 207 is a polypeptide including a region susceptible to an identified protease. Activity of the identified protease to cleave the reporter indicates the disease. Reporter 207 includes a cleavable substrate 221 connected to detectable analyte 210. When a cleavage by the identified protease occurs upon cleavable substrate 221, detectable analyte 210 is released from activity sensor 200 and may pass out of the tissue, excreted from the body and detected.

In various embodiments, activity sensors may include cyclic peptides that are structurally resistant to non-specific proteolysis and degradation in the body. Cyclic peptides can include protease-specific substrates or pH-sensitive bonds that allow the otherwise non-reactive cyclic peptide to release a reactive reporter molecule in response to the presence of the enzymes discussed herein. Cyclic peptides can require cleavage at a plurality of cleavage sites to increase specificity. The plurality of sites can be specific for the same or different proteases. Polycyclic peptides can be used comprising 2, 3, 4, or more cyclic peptide structures with various combinations of enzymes or environmental conditions required to linearize or release the functional peptide or other molecule. Cyclic peptides can include depsipeptides wherein hydrolysis of one or more ester bonds releases the linearized peptide. Such embodiments can be used to tune the timing of peptide release in environments such as plasma.

FIG. 3 shows an exemplary cyclic peptide 301 having a protease-specific substrate 309 and a stable cyclization linker 303. The protease-specific substrate 309 may comprise any number of amino acids in any order. For example, X₁ may be glycine. X₂ may be serine. X₃ may be aspartic acid. X₄ may be phenylalanine. X₅ may be glutamic acid. X₆ may be isoleucine. The N-terminus and C-terminus, coupled to the cyclization linker 303 comprise cyclization residues 305. The peptide may be engineered to address considerations such as protease stability, steric hindrance around cleavage site, macrocycle structure, and rigidity/flexibility of peptide chain. The type and number of spacer residues 307 can be chosen to address and alter many of those properties by changing the spacing between the various functional sites of the cyclic peptide. The cyclization linker and the positioning and choice of cyclization residues can also impact the considerations discussed above. Tuning domains such as PEG and/or reporters such as FAM can be included in the cyclic peptide.

The biological sample may be any sample from a subject in which the reporter may be detected. For example, the sample may be a tissue sample (such as a blood sample, a hard tissue sample, a soft tissue sample, etc.), a urine sample, saliva sample, mucus sample, fecal sample, seminal fluid sample, or cerebrospinal fluid sample.

Reporter molecules, released from activity sensors of the invention, may be detected by any suitable detection method able to detect the presence of quantity of molecules within the detectable analyte, directly or indirectly. For example, reporters may be detected via a ligand binding assay, which is a test that involves binding of the capture ligand to an affinity agent. Reporters may be directly detected, following capture, through optical density, radioactive emissions, or non-radiative energy transfers. Alternatively, reporters may be indirectly detected with antibody conjugates, affinity columns, streptavidin-biotin conjugates, PCR analysis, DNA microarray, or fluorescence analysis.

A ligand binding assay often involves a detection step, such as an ELISA, including fluorescent, colorimetric, bioluminescent and chemiluminescent ELISAs, a paper test strip or lateral flow assay, or a bead-based fluorescent assay.

In one example, a paper-based ELISA test may be used to detect the liberated reporter in urine. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Urine from the urine sample taken from the subject may then be added to the test spots, then streptavidin alkaline phosphate (ALP) conjugate may be added to the test spots, as the detection antibody. Bound ALP may then be exposed to a color reacting agent, such as BCIP/NBT (5-bromo-4-chloro-3′-indolyphosphate p-toluidine salt/nitro-blue tetrazolium chloride), which causes a purple colored precipitate, indicating presence of the reporter.

In another example, volatile organic compounds may be detected by analysis platforms such as gas chromatography instrument, a breathalyzer, a mass spectrometer, or use of optical or acoustic sensors.

Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

A modified breathalyzer instrument may also be used to detect volatile organic compounds. In a traditional breathalyzer that is used to detect an alcohol level in blood, a subject exhales into the instrument, and any ethanol present in the subject's breath is oxidized to acetic acid at the anode. At the cathode, atmospheric oxygen is reduced. The overall reaction is the oxidation of ethanol to acetic acid and water, which produces an electric current that may be detected and quantified by a microcontroller. A modified breathalyzer instrument exploiting other reactions may be used to detect various volatile organic compounds.

Mass spectrometry may be used to detect and distinguish reporters based on differences in mass. In mass spectrometry, a sample is ionized, for example by bombarding it with electrons. The sample may be solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis may be performed directly on the biological sample or the detectable analyte may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis.

Detecting the detectable analyte may provide a qualitative assessment (e.g., whether the detectable analyte is present or absent) or a quantitative assessment (e.g., the amount of the detectable analyte present) to indicate a comparative activity level of the enzymes. The quantitative value may be calculated by any means, such as, by determining the percent relative amount of each fraction present in the sample. Methods for making these types of calculations are known in the art.

The detectable analyte may be labeled. For example, a label may be added directly to a nucleic acid when the isolated detectable analyte is subjected to PCR. For example, a PCR reaction performed using labeled primers or labeled nucleotides will produce a labeled product. Labeled nucleotides, such as fluorescein-labeled CTP are commercially available. Methods for attaching labels to nucleic acids are well known to those of ordinary skill in the art and, in addition to the PCR method, include, for example, nick translation and end-labeling.

Labels suitable for use in the reporter include any type of label detectable by standard methods, including spectroscopic, photochemical, biochemical, electrical, optical, or chemical methods. The label may be a fluorescent label. A fluorescent label is a compound including at least one fluorophore. Commercially available fluorescent labels include, for example, fluorescein phosphoramidites, rhodamine, polymethadine dye derivative, phosphores, Texas red, green fluorescent protein, CY3, and CY5.

Other known techniques, such as chemiluminescence or colormetrics (enzymatic color reaction), can also be used to detect the reporter. Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET), such as fluorescence resonance energy transfer (FRET). Cleavage of the peptide results in separation of the chromophore and fluorophore, removal of the quench, and generation of a subsequent signal measured from the donor fluorophore. Examples of FRET pairs include 5-Carboxyfluorescein (5-FAM) and CPQ2, FAM and DABCYL, Cy5 and QSY21, Cy3 and QSY7.

In various embodiments, the activity sensor may include ligands to aid it targeting particular tissues or organs. When administered to a subject, the activity sensor is trafficked in the body through various pathways depending on how it enters the body. For example, if activity sensor is administered intravenously, it will enter systemic circulation from the point of injection and may be passively trafficked through the body.

For the activity sensor to respond to enzymatic activity within a specific cell, at some point during its residence time in the body, the activity sensor must come into the presence of the enzyme and have an opportunity to be cleaved and linearized by the enzyme to release the linearized reporter or therapeutic molecule. From a targeting perspective, it is advantageous to provide the activity sensor with a means to target specific cells or a specific tissue type where such enzymes of interest may be present. To achieve this, ligands for receptors of the specific cell or specific tissue type may be provided as the tuning domains and linked to polypeptide.

Cell surface receptors are membrane-anchored proteins that bind ligands on the outside surface of the cell. In one example, the ligand may bind ligand-gated ion channels, which are ion channels that open in response to the binding of a ligand. The ligand-gated ion channel spans the cell's membrane and has a hydrophilic channel in the middle. In response to a ligand binding to the extracellular region of the channel, the protein's structure changes in such a way that certain particles or ions may pass through. By providing the activity sensor with tuning domains that include ligands for proteins present on the cell surface, the activity sensor has a greater opportunity to reach and enter specific cells to detect enzymatic activity within those cells.

By providing the activity sensor with tuning domains, distribution of the activity sensor may be modified because ligands may target the activity sensor to specific cells or specific tissues in a subject via binding of the ligand to cell surface proteins on the targeted cells. The ligands of tuning domains may be selected from a group including a small molecule; a peptide; an antibody; a fragment of an antibody; a nucleic acid; and an aptamer.

Once activity sensor reaches the specific tissue, ligands may also promote accumulation of the activity sensor in the specific tissue type. Accumulating the activity sensor in the specific tissue increases the residence time of the activity sensor and provides a greater opportunity for the activity sensor to be enzymatically cleaved by proteases in the tissue, if such proteases are present.

When the activity sensor is administered to a subject, it may be recognized as a foreign substance by the immune system and subjected to immune clearance, thereby never reaching the specific cells or specific tissue where the specific enzymatic activity can release the therapeutic compound or reporter molecule. Furthermore, generation of an immune response can defeat the purpose of immune-response-sensitive activity sensors. To inhibit immune detection, it is preferable to use a biocompatible carrier so that it does not elicit an immune response, for example, a biocompatible carrier may include one or more subunits of polyethylene glycol maleimide. Further, the molecular weight of the polyethylene glycol maleimide carrier may be modified to facilitate trafficking within the body and to prevent clearance of the activity sensor by the reticuloendothelial system. Through such modifications, the distribution and residence time of the activity sensor in the body or in specific tissues may be improved.

In various embodiments, the activity sensor may be engineered to promote diffusion across a cell membrane. As discussed above, cellular uptake of activity sensors has been well documented. See Gang. Hydrophobic chains may also be provided as tuning domains to facilitate diffusion of the activity sensor across a cell membrane may be linked to the activity sensor.

The tuning domains may include any suitable hydrophobic chains that facilitate diffusion, for example, fatty acid chains including neutral, saturated, (poly/mono) unsaturated fats and oils (monoglycerides, diglycerides, triglycerides), phospholipids, sterols (steroid alcohols), zoosterols (cholesterol), waxes, and fat-soluble vitamins (vitamins A, D, E, and K).

In some embodiments, the tuning domains include cell-penetrating peptides. Cell-penetrating peptides (CPPs) are short peptides that facilitate cellular intake/uptake of activity sensors of the disclosure. CPPs preferably have an amino acid composition that either contains a high relative abundance of positively charged amino acids such as lysine or arginine or has sequences that contain an alternating pattern of polar/charged amino acids and non-polar, hydrophobic amino acids. See Milletti, 2012, Cell-penetrating peptides: classes, origin, and current landscape, Drug Discov Today 17:850-860, incorporated by reference. Suitable CPPs include those known in the literature as Tat, R6, R8, R9, Penetratin, pVEc, RRL helix, Shuffle, and Penetramax. See Kristensen, 2016, Cell-penetrating peptides as tools to enhance non-injectable delivery of biopharmaceuticals, Tissue Barriers 4(2):e1178369, incorporated by reference.

In certain embodiments, an activity sensor may include a biocompatible polymer as a tuning domain to shield the activity sensor from immune detection or inhibit cellular uptake of the activity sensor by macrophages.

When a foreign substance is recognized as an antigen, an antibody response may be triggered by the immune system. Generally, antibodies will then attach to the foreign substance, forming antigen-antibody complexes, which are then ingested by macrophages and other phagocytic cells to clear those foreign substances from the body. As such, when an activity sensor enters the body, it may be recognized as an antigen and subjected to immune clearance, preventing the activity sensor from reaching a specific tissue to detect enzymatic activity. To inhibit immune detection of the activity sensor, for example, PEG tuning domains may be linked to the activity sensor. PEG acts as a shield, inhibiting recognition of the activity sensor as a foreign substance by the immune system. By inhibiting immune detection, the tuning domains improve the residence time of the activity sensor in the body or in a specific tissue.

Enzymes have a high specificity for specific substrates by binding pockets with complementary shape, charge and hydrophilic/hydrophobic characteristic of the substrates. As such, enzymes can distinguish between very similar substrate molecules to be chemoselective (i.e., preferring an outcome of a chemical reaction over an alternative reaction), regioselective (i.e., preferring one direction of chemical bond making or breaking over all other possible directions), and stereospecific (i.e., only reacting on one or a subset of stereoisomers).

Steric effects are nonbonding interactions that influence the shape (i.e., conformation) and reactivity of ions and molecules, which results in steric hindrance. Steric hindrance is the slowing of chemical reactions due to steric bulk, affecting intermolecular reactions. Various groups of a molecule may be modified to control the steric hindrance among the groups, for example to control selectivity, such as for inhibiting undesired side-reactions. By providing the activity sensor with tuning domains such as spacer residues between the carrier and the cleavage site and/or any bioconjugation residue, steric hindrance among components of activity sensor may be minimized to increase accessibility of the cleavage site to specific proteases. Alternatively, steric hindrance can be used as described above to prevent access to the cleavage site until an unstable cyclization linker (e.g., an ester bond of a cyclic depsipeptide) has degraded. Such unstable cyclization linkers can be other known chemical moieties that hydrolyze in defined conditions (e.g., pH or presence of a certain analyte) which may be selected to respond to specific characteristics of a target environment.

In various embodiments, activity sensors may include D-amino acids aside from the target cleavage site to further prevent non-specific protease activity. Other non-natural amino acids may be incorporated into the peptides, including synthetic non-native amino acids, substituted amino acids, or one or more D-amino acids.

In some embodiments, tuning domains may include synthetic polymers such as polymers of lactic acid and glycolic acid, polyanhydrides, polyurethanes, and natural polymers such as alginate and other polysaccharides including dextran and cellulose, collagen, albumin and other hydrophilic proteins, zein and other prolamines and hydrophobic proteins, copolymers and mixtures thereof.

One of skill in the art would know what peptide segments to include as protease cleavage sites in an activity sensor of the disclosure. One can use an online tool or publication to identify cleavage sites. For example, cleavage sites are predicted in the online database PROSPER, described in Song, 2012, PROSPER: An integrated feature-based tool for predicting protease substrate cleavage sites, PLoSOne 7(11):e50300, incorporated by reference. Any of the compositions, structures, methods or activity sensors discussed herein may include, for example, any suitable cleavage site, as well as any further arbitrary polypeptide segment to obtain any desired molecular weight. To prevent off-target cleavage, one or any number of amino acids outside of the cleavage site may be in a mixture of the D and/or the L form in any quantity.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of providing personalized treatment to a patient comprising:
administering an activity sensor cocktail to a patient;
analyzing results obtained from administration of the activity sensor cocktail;
accessing data obtained from at least one other source; and
determining a personalized course of treatment for the patient based on analysis of results from activity sensor cocktail administration and the data obtained from the at least one other source.

2. The method of claim 1, wherein the activity sensor cocktail comprises a plurality of activity sensors each comprising:
a carrier comprising one or a plurality of molecular subunits; and
a plurality of detectable reporters, each linked to the carrier by a cleavable linker containing the cleavage site of an enzyme, wherein the activity sensor reports activity of one or more enzymes by releasing the reporters upon cleavage by the one or more enzymes.

3. The method of claim 1, wherein the determining step comprises diagnosing a disease.

4. The method of claim 1, wherein the determining step comprises identifying a stage in a disease progression.

5. The method of claim 1, wherein the determining step comprises predicting a response to a therapeutic treatment.

6. The method of claim 1, wherein the at least one other source comprises electronic medical records (EMR).

7. The method of claim 1, wherein the at least one other source comprise molecular diagnostic data.

8. The method of claim 7, wherein the molecular diagnostic data is selected from the group consisting of nucleic acid sequence information, epigenetic information, DNA methylation, and RNA expression data.

9. The method of claim 1, wherein the at least one other source comprises comorbidity information.

10. The method of claim 1, wherein determining step comprises identifying patterns in the results from the activity sensor cocktail administration and the data obtained from the at least one other source indicative of an outcome.

11. The method of claim 10, wherein the patterns are identified through machine learning analysis of data for patients with known outcomes.

12. The method of claim 1, wherein the determining step is performed by a computer comprising a tangible, non-transitory memory coupled to a processor.

13. A method for identifying diagnostic indicators in patient data, the method comprising:
analyzing results obtained from administration of an activity sensor cocktail to a plurality of patients with known outcomes;
accessing data for the plurality of patients obtained from at least one other source; and
providing the known outcomes, the results, and the data to a machine learning system;
identifying, through machine learning analysis, patterns in the results and the data indicative of one or more of the known outcomes using the machine learning system.

14. The method of claim 13, wherein the activity sensor cocktail comprises a plurality of activity sensors each comprising:
a carrier comprising one or a plurality of molecular subunits; and
a plurality of detectable reporters, each linked to the carrier by a cleavable linker containing the cleavage site of an enzyme, wherein the activity sensor reports activity of one or more enzymes by releasing the reporters upon cleavage by the one or more enzymes.

15. The method of claim 13, wherein the known outcomes comprise development of a disease.

16. The method of claim 13, wherein the known outcomes comprise progression of a disease.

17. The method of claim 13, wherein the known outcomes comprise a response to a therapeutic treatment.

18. The method of claim 13, wherein the at least one other source comprises electronic medical records (EMR).

19. The method of claim 13, wherein the at least one other source comprises comorbidity information.

20. The method of claim 13, wherein the at least one other source comprise molecular diagnostic data.
